(19) United States
(12) Patent Application Publication
(10) Pub. No.: US 2008/0147029 A1
(43) Pub. Date: Jun. 19, 2008

(54) Absorbent articles containing an odor control agent that immobilizes odor producing bacteria

(76) Inventors: Courtney E. Pate, Appleton, WI (US); Amanda M. Barker Furtado, Appleton, WI (US); Curtis N. Sayre, Atlanta, GA (US)

Correspondence Address: DORITY & MANNING, P.A., POST OFFICE BOX 1449, GREENVILLE, SC, 29602-1449, US

(21) Appl. No.: 11/640,128

(22) Filed: Dec. 15, 2006

(51) Int. Cl. A61F 13/15

(52) U.S. Cl. 604/360

(57) ABSTRACT

Absorbent articles are disclosed containing an odor control agent. The odor control agent, for instance, may be incorporated into an absorbent core present in the absorbent article. The absorbent article may be, for instance, a diaper, a feminine hygiene product, an adult incontinence product, a training pant, and the like. In accordance with the present disclosure, the odor control agent comprises a chemical compound having a positive charge. For instance, in one embodiment, the chemical compound comprises aluminum chlorohydrate. The odor control agent is capable of immobilizing bacteria that cause odors. By immobilizing the bacteria, the bacteria are not capable of multiplying and are therefore not capable of producing odor producing substances.

### BACKGROUND

Personal care products, such as adult incontinent products, diapers, training pants, and feminine hygiene products, have numerous sources of odors during and after use. For instance, many odors are created as a result of naturally occurring bacteria on the skin flora. In the past, various attempts have been made to incorporate into personal care products odor control agents that are intended to absorb odors or fragrances intended to mask odors.

Although advances have been made in controlling odors emanating from personal care products, improvements are still needed. In particular, an odor control agent is needed that can be incorporated into a personal care product and that can bind or immobilize bacteria that cause odors.

### SUMMARY

In general, the present disclosure is directed to an absorbent article having odor control properties. The article, for instance, may comprise an absorbent core that includes cellulosic fibers, such as pulp fibers. In accordance with the present disclosure, the cellulosic fibers are treated with an odor control agent. The odor control agent comprises a chemical compound having a net positive charge in an amount sufficient to bind and immobilize odor producing bacteria.

The chemical compound, for instance, in one embodiment, may comprise a cationic polymer, a cationic oligomer, or mixtures thereof. For instance, the chemical compound may comprise an aluminum oligomer or an aluminum salt. Particular examples of such compounds may include, for instance, aluminum chlorohydrate, aluminum chlorohydrol, aluminum potassium sulfate (alum), aluminum chloride, polyaluminum chloride, and mixtures thereof. In one embodiment, the odor control agent may comprise particles coated with one of the cationic polymers or cationic oligomers described above.

The amount that the odor control agent is incorporated into the absorbent core may depend upon various factors. For instance, the amount the odor control agent is incorporated into the absorbent core may depend upon the particular article being formed and the desired result. In one embodiment, for instance, the odor control agent may be incorporated into the absorbent core in an amount from about 0.01% to about 10% by weight, such as from about 0.5% to about 5% by weight.

The absorbent article made in accordance with the present disclosure may comprise any suitable incontinent article. For instance, the absorbent core of the absorbent article may be positioned in between an outer cover material and a body side liner. The absorbent article may comprise, for instance, a diaper, a feminine hygiene product, a training pant, a swim pant, an adult incontinence article, or the like.

The odor control agent can be incorporated into the absorbent core according to various methods. In one embodiment, for instance, a fibrous sheet containing pulp fibers may be treated with an aqueous solution containing the odor control agent. Once treated, the sheet may be fiberized to form a plurality of individual pulp fibers. The fibers can then be entrained in a gaseous stream in order to form an airformed web. The airformed web can then be incorporated into an absorbent article as desired. In one embodiment, the airformed web can include superabsorbent particles in addition to the pulp fibers.

Other features and aspects of the present disclosure are discussed in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the following figures:

FIG. 1 is a schematic view of one embodiment of a forming apparatus that may be used in the present disclosure; and

FIG. 2 illustrates a perspective of an absorbent article that may be formed according to one embodiment of the present disclosure.

Repeat use of reference characters in the present specification and drawings is intended to represent same or analogous features or elements of the disclosure.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to an absorbent article having odor control properties. The absorbent article may be, for instance, any suitable personal care product such as an adult incontinence product, feminine hygiene product, a diaper, a training pant, a swim pant, and the like. In one embodiment, for instance, the absorbent article includes an outer cover material, a body side liner, and an absorbent core positioned in between the outer cover and the body side liner. In accordance with the present disclosure, an odor control agent is incorporated into the absorbent core. The odor control agent comprises a chemical compound having a net positive charge in an amount sufficient to bind and immobilize odor producing components, such as bacteria.

When an absorbent article is worn and contacted with the skin and/or insulted with a body fluid, various different species, such as bacteria, are transferred to the absorbent article that cause odors. For example, one odorous molecule that is often found in absorbent products is ammonia. Ammonia is generated from the hydrodolysis of urea. More particularly, the production of ammonia results from the following reaction:

(NH₂)2CO+H₂O+Urease→CO₂+2HNH₃

As shown above, the enzyme urease can react with urea found in urine to produce ammonia. The enzyme urease is produced by bacteria that are also transferred into the absorbent product.

In addition to ammonia, various other odorous molecules can also be produced in absorbent articles. For instance, other bacteria typically found in insulted absorbent articles can also produce other odorous compounds such as methylsulfide, 3-methyl-1-butanol, dimethyl disulfide, 4-heptanone, pyrazine, and the like.

According to the present disclosure, an odor control agent is incorporated into the absorbent article that has bacteriostatic properties. In particular the odor control agent is capable of attracting and/or trapping bacteria in the article. Once trapped by the odor control agent, the bacteria is prevented from reproducing and is prevented from producing the above described odorous compounds. By inhibiting or limiting the production of these compounds via binding of the bacteria and limiting the growth of the bacteria, it is possible to decrease or eliminate the overall odor associated with an absorbent article. For instance, in one embodiment, the odor control agent is capable of binding to bacteria that produce urease or by limiting the production of the urease enzyme and the production of ammonia.

The odor control agent of the present disclosure generally comprises a chemical compound having a net positive charge. By having a net positive charge, the odor control agent can attract and trap negatively charged matter through the application of physical means and Colulombic attraction, without the use of harsh chemicals. For example, the odor control agent can provide a positive charge to the absorbent article that can electrostatically attract and/or trap negatively charged matter, such as molecules, bacteria, allergens, particles, microbes, cells, fungi, anions, other microorganisms, pathogens, and the like. Once trapped by the odor control agent, the reproduction and growth of bacteria is hindered within the article.

As will be described in greater detail below, in one embodiment, the odor control agent can also interact, such as chemically, electrostatically, or physically with fibers and other matter present within the absorbent article. For instance, the odor control agent can be attached or bound with cellulosic fibers such as, fluff pulp fibers that may be present in the absorbent article.

As described above, the odor control agent of the present disclosure generally comprises a chemical compound having a net positive charge. The chemical compound, for instance, may comprise a cationic polymer, a cationic oligomer, a cationic salt, or mixtures thereof. The particular odor control agent used in any specific product may depend upon the particular application and the desired result.

Examples of chemical compounds that may be used in the present disclosure include aluminum polymers, aluminum oligomers, and aluminum salts. For instance, in one embodiment, the odor control agent may comprise aluminum chlorohydrate, aluminum chlorohydrol, aluminum potassium sulfate (alum), aluminum chloride, polyaluminum chloride, and mixtures thereof. Aluminum chlorohydrate, for instance, may be represented as follows:

Al₂(OH)₆₋ₙClₙ,

wherein n can be from 1-5.

In still another embodiment, the odor control agent may comprise particles coated with a cationic material. For instance, the odor control agent may contain nanoparticles coated with any of the chemical compounds described above, such as an aluminum oligomer. The nanoparticles may comprise, for instance, silica particles or alumina particles.

Of particular advantage, many of the materials described above are also capable of bonding to cellulosic fibers, such as pulp fibers that may be present in an absorbent core of an absorbent article. The odor control agent can be combined with the materials used to form the absorbent core using any suitable method or technique. For example, in one embodiment, cellulosic fibers to be present in the absorbent core can be contacted with a solution containing the odor control agent. The fibers, for instance, can be saturated with the solution. After saturation, the fibers can be dried allowing the odor control agent to remain integral to the fibers. Depending upon the odor control agent used, for instance, the odor control agent can ionically bond or covalently bond to the fibers. In alternative embodiments, a solution containing the odor control agent can be sprayed or printed onto cellulosic fibers. The fibers can be contacted with the solution in any suitable form. For instance, the fibers can be contacted with the solution while the fibers are in an organized web or bat or can be combined with the solution when in an individual state.

The odor control agent of the present disclosure may be combined with other odor control compositions if desired. For instance, the odor control agent of the present disclosure may be combined with other compositions that absorb odors as opposed to having bacteriostatic properties. For example, other compositions that may be combined with the odor control agent include inorganic oxide particles, such as silica, alumina, zirconia, magnesium oxide, titanium dioxide, iron oxide, zinc oxide, copper oxide, zeolites, clays (e.g., smectite clay), combinations thereof, and so forth, may be employed in some embodiments of the present disclosure. Various examples of such inorganic oxide particles are described in U.S. Patent Application Publication Nos. 2003/0203009 to MacDonald; 2005/0084412 to Macdonald, et al.; 2005/0085144 to Macdonald, et al.; 2005/0084464 to McGrath, et al.; 2005/0084474 to Wu, et al.; 2005/0084438 to Do, et al.; and 2006/0008442 to MacDonald, et al., which are incorporated herein in their entirety by reference thereto for all purposes.

In one embodiment, for instance, an inorganic oxide particle may be used that is modified with one or more transition metals. The transition metal may comprise copper, manganese, iron, cobalt, nickel, zinc and mixtures thereof.

Other compositions that may be combined with the odor control agent include quinone powders having odor control characteristics and/or activated carbon particles.

Various different types of materials may be treated with the odor control agent in accordance with the present disclosure. For instance, in one embodiment, hydrophilic fibers may be treated and used to produce an absorbent core in an absorbent product. Hydrophilic fibers may include natural and/or synthetic fluff pulp fibers. The fluff pulp fibers may be kraft pulp, sulfite pulp, thermomechanical pulp, etc. In addition, the fluff pulp fibers may include high-average fiber length pulp, low-average fiber length pulp, or mixtures of the same. One example of suitable high-average length fluff pulp fibers includes softwood kraft pulp fibers. Softwood kraft pulp fibers are derived from coniferous trees and include pulp fibers such as, but not limited to, northern, western, and southern softwood species, including redwood, red cedar, hemlock, Douglas-fir, true firs, pine (e.g., southern pines), spruce (e.g., black spruce), combinations thereof, and so forth. Northern softwood kraft pulp fibers may be used in the present disclosure. One example of commercially available southern softwood kraft pulp fibers suitable for use in the present disclosure, include those available from Weyerhaeuser Company with offices in Federal Way, Wash. under the trade designation of “NB-416.” Another suitable fluff pulp for use in the present disclosure is a bleached, sulfate wood pulp containing primarily softwood fibers that is available from Bowater Corp. with offices in Greenville, S.C. under the trade name CoosAbsorb S pulp. Low-average length fibers may also be used in the present disclosure. An example of suitable low-average length pulp fibers is hardwood kraft pulp fibers. Hardwood kraft pulp fibers are derived from deciduous trees and include pulp fibers such as, but not limited to, eucalyptus, maple, birch, aspen, etc. Eucalyptus kraft pulp fibers may be particularly desired to increase softness, enhance brightness, increase opacity, and change the pore structure of the sheet to increase its wicking ability.

In addition, synthetic fibers may also be utilized. Some suitable polymers that may be used to form the synthetic fibers include, but are not limited to: polyolefins, such as, polyethylene, polypropylene, polybutylene, and so forth; polyesters, such as polyethylene terephthalate, poly(glycolic acid) (PGA), poly(lactic acid) (PLA), poly(β-malic acid) (PMLA), poly(ε-caprolactone) (PCL), poly(ρ-dioxanone) (PDS), poly(3-hydroxybutyrate) (PHB), and so forth; and, polyamides, such as nylon and so forth. Synthetic or natural cellulosic polymers, including but not limited to, cellulosic esters; cellulosic ethers; cellulosic nitrates; cellulosic acetates; cellulosic acetate butyrates; ethyl cellulose; regenerated celluloses, such as viscose, rayon, and so forth. Non-wood fibers may also be used, including fiber originating from hemp, straw, flax, bagasse, and mixtures thereof may be used in the present disclosure.

In one particular embodiment, the odor control agent may be topically applied to a dry lap sheet of fluff pulp. The dry lap sheet may then be subjected to a fiberizing process that breaks the sheet into a plurality of individual fibers. These individual fibers may then be supplied to a forming chamber that deposits the fibers onto a foraminous surface to form an airformed web. Due to the vigorous mixing imparted by the fiberizing process, the odor control agent thus becomes homogeneously distributed throughout the web structure. Additionally, a superabsorbent material, that also optionally may be precombined with the odor control agent, may be separately injected into the forming chamber where it will mix with the fibers.

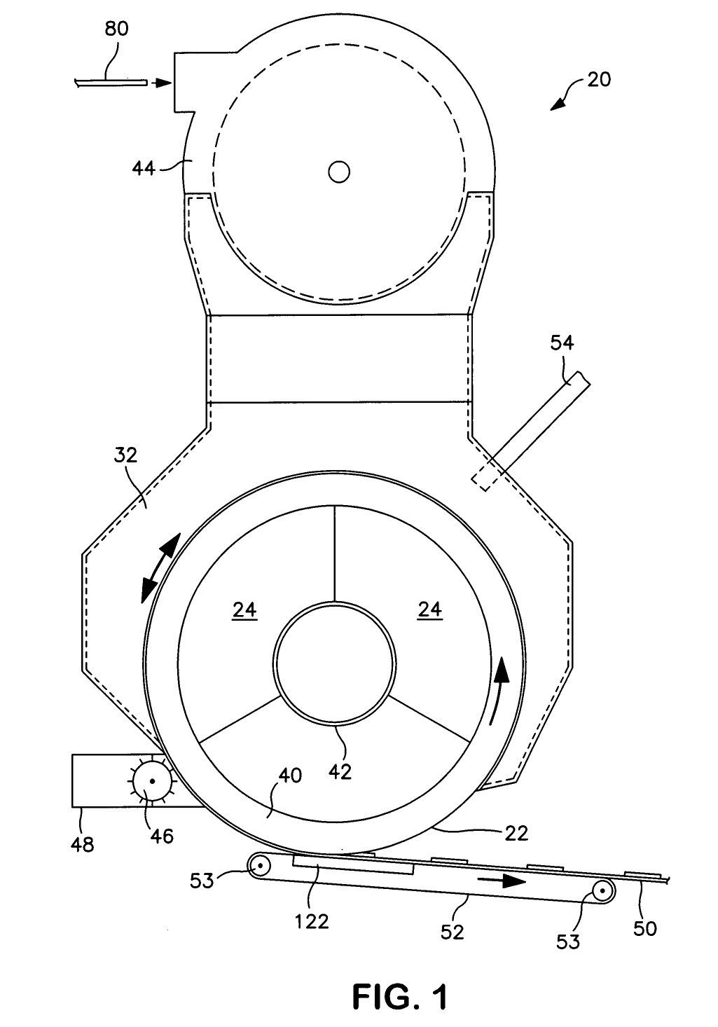

Various specific embodiments of the aforementioned techniques will now be described in greater detail. It should be understood, however, that the embodiments described are merely exemplary, and that various other embodiments are also contemplated by the present disclosure. In this regard, referring to FIG. 1, one embodiment of a forming apparatus 20 that may be used in the present disclosure is depicted. As shown, the apparatus 20 includes a fiberizer 44, such as a rotary hammermill, rotatable picker roll, or any other conventional fiberizing device. A dry lap sheet 80 of fluff pulp is supplied to the fiberizer 44 to form a plurality of individual fibers. In one embodiment of the present disclosure, the dry lap sheet is pre-treated with the odor control agent using conventional techniques, such as printing, dipping, spraying, and so forth. The percent coverage and add-on level of the odor control agent be selectively varied to achieve any desired distribution within the final web. For instance, the percent coverage of the odor control agent may be greater than about 50%, in some embodiments greater than about 80%, and in some embodiments, approximately 100% of the area of a given surface. Likewise, the odor control agent may be applied in an amount from about 0.01 wt. % to about 10 wt. %, and in some embodiments, from about 0.1 wt. % to about 5 wt. % based on the weight of fibers.

Once fiberized, the individual fibers are entrained in a stream of a gaseous medium (e.g., air), and then ejected or otherwise introduced into a forming chamber 32. Although illustrated below the fiberizer 44, the forming chamber 32 may also be positioned at any other location, such as to the side or at a remote location that is spaced away from the fiberizer 44. The forming chamber 32 may direct and concentrate the gas-entrained fibers and provide a desired velocity profile in the stream. Various suitable forming chamber configurations are described, for instance, in U.S. Pat. Nos. 4,927,582 to Bryson and 6,630,096 to Venturino, et al., which are incorporated herein in their entirety by reference thereto for all purposes.

The apparatus 20 also includes a movable, foraminous forming surface 22 onto which a fibrous web 50 is formed. Typically, the forming surface 22 is provided by a foraminous, air permeable material, such as a wire forming cloth, screen, perforated plate, fabric, belt, drum, etc. Suitable forming belts are commercially available from the Paper Converting Machine Co. of Green Bay, Wis. and from Curt G. Joa, Inc. of Sheboygan Falls, Wis. In the illustrated embodiment, the forming surface 22 is provided by a forming drum 40. During use, the forming drum 40 is rotated in a desired direction by a drum drive shaft (not shown) that is operatively joined to a drive mechanism (not shown). The drive mechanism may include an electric or other motor that is directly or indirectly coupled to the drive shaft. If desired, the forming surface 22 may include a series of separately removable, forming sections that are distributed circumferentially along the periphery of the forming drum 40. Such forming sections may optionally provide a selected repeat pattern that is formed in the resulting fibrous web. The repeat pattern may correspond to a desired shape of an individual absorbent pad that is intended for assembly or other placement in an absorbent article. Suitable forming drum systems are described in more detail in U.S. Pat. Nos. 4,666,647 to Enloe, et al.; 4,761,258 to Enloe; and 6,330,735 to Hahn, et al., which are incorporated herein in their entirety by reference thereto for all purposes.

The interior space of the forming drum 40 may include a vacuum zone having the general form of an arcuate segment located at the portion of the forming surface 22 positioned within the forming chamber 32. In the illustrated embodiment, for instance, the vacuum zone is located adjacent to the forming chamber 32 and may include features provided by a vacuum duct 24. The portion of the forming drum 40 positioned within the boundaries of the forming chamber 32 may delimit or otherwise provide a lay-down zone of the forming surface 22. Such a vacuum lay-down zone may, for example, constitute a circumferential, cylindrical surface portion of the rotatable drum 40. A pressure differential is imposed on the surface of the vacuum lay-down zone by a conventional vacuum source (e.g., vacuum pump, an exhaust blower, etc.) to provide a relatively low pressure under the forming surface 22. Under the influence of the vacuum source, a conveying gas stream is thus drawn through the forming surface 22 into the interior of the forming drum 40, and subsequently passed out of the drum through a vacuum supply conduit 42. As the gas-entrained fibers impinge on the foraminous forming surface 22, a gas (e.g., air) is passed through the forming surface 22 to cause the fibers to be retained on the surface to form a fibrous web 50.

If desired, drum rotation may then pass the fibrous web 50 to a scarfing zone where excess thickness may be trimmed and removed to a predetermined extent. More specifically, the scarfing system may include a scarfing chamber 48 and a scarfing roll 46 positioned therein. The scarfing roll 46 rotates in a direction opposite to the movement direction of the laid fibrous web 50. Alternatively, the scarfing roll 46 may be rotated to provide a co-directional movement of the roller surface relative to the surface of the forming drum proximate thereto. In either case, the rotational speed of the scarfing roll 46 is selected to provide an effective scarfing action against the contacted surface of the formed fibrous web 50 to abrade excess fibrous material therefrom. If desired, the removed fibrous material may be recycled back into the forming chamber 32 or the fiberizer 44, as desired. Additionally, the scarfing roll 46 may rearrange and redistribute the web material along the longitudinal machine-direction of the web and/or along the lateral cross-direction of the web.

After the scarfing operation, the portion of the forming surface 22 that is carrying the fibrous web 50 may be moved to an optional pressure blow-off zone of the forming drum system. In the blow-off zone, a gas (e.g., air) may be introduced under pressure and directed radially outwardly against the fibrous web on the portion of the forming surface that becomes aligned with the blow-off zone. The gas pressure may cause the fibrous web to release from the forming surface 22 onto a suitable web transport mechanism. The web transporter may receive the fibrous web 50 from the forming drum 40 and convey it for further processing. Suitable web transporters may include conveyer belts, vacuum drums, transport rollers, electromagnetic suspension conveyors, fluid suspension conveyors or the like, as well as combinations thereof. As shown, for instance, the web transporter may include a conveyor belt 52 disposed about rollers 53. In a particular embodiment, a vacuum suction box 122 is located below the conveyor belt 52 to help remove the web 50 from the forming surface 22. The vacuum box 122 opens onto the belt 52 and draws gas through perforations in the conveyor belt 52. In turn, this flow of gas draws the web 50 away from the forming surface 22. The vacuum box 122 may be employed with or without the use of a positive pressure in the blow-off zone. The removed fibrous web 50 may provide an interconnected series of pads, and each pad may have a selected surface contour that substantially matches the contour provided by the various, corresponding portions of the forming surface 22 upon which each individual pad was formed.

It will be readily apparent that various conventional devices and techniques may be employed to further process the web 50. For example, various conventional devices and techniques may be employed to sever fibrous web 50 into predetermined lengths to provide selected laid fibrous articles. The severing system may, for example, include a die cutter, a water cutter, a rotary knives, reciprocating knives, energy beam cutters, particle beam cutters or the like, as well as combinations thereof. After severing, the discrete fibrous pads 50 may be transported and delivered for further processing operations.

As described above, a superabsorbent material may also be introduced into the forming chamber 32 during production of the web 50. In one embodiment, for instance, superabsorbent particles can be introduced into the forming chamber 32 using a nozzle 54. The superabsorbent particles may be present in the resulting fibrous web in an amount from about 0% up to about 90% by weight.

When formed in accordance with the present disclosure, the resulting fibrous web contains an odor control agent that is capable of reducing or inhibiting one or more odors. Such an odor-reducing web may be used in a wide variety of applications. In one particular embodiment, for example, the fibrous web may be used in an absorbent article. An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth. Materials and processes suitable for forming such absorbent articles are well known to those skilled in the art. Typically, absorbent articles include a substantially liquid-impermeable layer (e.g., outer cover), a liquid-permeable layer (e.g., bodyside liner, surge layer, etc.), and an absorbent core. The treated fibrous web of the present disclosure may be employed as any one or more of the liquid transmissive (non-retentive) and absorbent layers, and is desirably used to form the absorbent core. For example, the treated fibrous web may form the entire absorbent core. Alternatively, the treated fibrous web may form only a portion of the core, such as a layer of an absorbent composite that includes one or more additional layers (e.g., wet-formed paper webs, coform webs, etc.). Regardless, the odor control agent contained within the fibrous web can immobilize and trap malodorous producing bacteria absorbed by the absorbent core and thereby reduce the amount of odor released by the article. Bacteria that can be immobilized by the odor control agent of the present disclosure include, for instance, P. Mirablis, E. Faecalis, E. Coli, K. Pnuemonae, and the like.

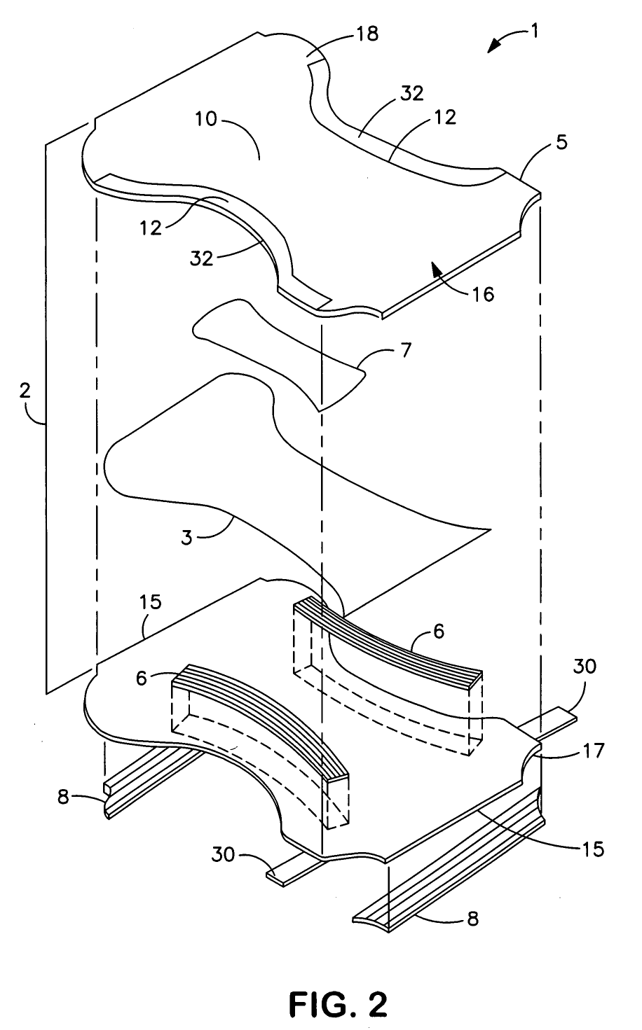

Various embodiments of an absorbent article that may be formed according to the present disclosure will now be described in more detail. For purposes of illustration only, an absorbent article is shown in FIG. 2 as a diaper 1. However, as noted above, the disclosure may be embodied in other types of absorbent articles, such as incontinence articles, sanitary napkins, diaper pants, feminine napkins, children's training pants, and so forth. In the illustrated embodiment, the diaper 1 is shown as having an hourglass shape in an unfastened configuration. However, other shapes may of course be utilized, such as a generally rectangular shape, T-shape, or I-shape. As shown, the diaper 1 includes a chassis 2 formed by various components, including an outer cover 17, bodyside liner 5, absorbent core 3, and surge layer 7. It should be understood, however, that other layers may also be used in the present disclosure. Likewise, one or more of the layers referred to in FIG. 2 may also be eliminated in certain embodiments.

The outer cover 17 is typically formed from a material that is substantially impermeable to liquids. For example, the outer cover 17 may be formed from a thin plastic film or other flexible liquid-impermeable material. In one embodiment, the outer cover 17 is formed from a polyethylene film. If a more cloth-like feeling is desired, the outer cover 17 may be formed from a polyolefin film laminated to a nonwoven web. For example, a stretch-thinned polypropylene film may be thermally laminated to a spunbond web of polypropylene fibers.

The diaper 1 also includes a bodyside liner 5. The bodyside liner 5 is generally employed to help isolate the wearer's skin from liquids held in the absorbent core 3. For example, the liner 5 presents a bodyfacing surface that is typically compliant, soft feeling, and non-irritating to the wearer's skin. Typically, the liner 5 is also less hydrophilic than the absorbent core 3 so that its surface remains relatively dry to the wearer. The liner 5 may be liquid-permeable to permit liquid to readily penetrate through its thickness. The bodyside liner 5 may be formed from a wide variety of materials, such as porous foams, reticulated foams, apertured plastic films, natural fibers (e.g., wood or cotton fibers), synthetic fibers (e.g., polyester or polypropylene fibers), or a combination thereof. In some embodiments, woven and/or nonwoven fabrics are used for the liner 5. For example, the bodyside liner 5 may be formed from a meltblown or spunbonded web of polyolefin fibers. The liner 5 may also be a bonded-carded web of natural and/or synthetic fibers. The liner 5 may further be composed of a substantially hydrophobic material that is optionally treated with a surfactant or otherwise processed to impart a desired level of wettability and hydrophilicity.

As illustrated in FIG. 2, the diaper 1 may also include a surge layer 7 that helps to decelerate and diffuse surges or gushes of liquid that may be rapidly introduced into the absorbent core 3. Desirably, the surge layer 7 rapidly accepts and temporarily holds the liquid prior to releasing it into the storage or retention portions of the absorbent core 3. In the illustrated embodiment, for example, the surge layer 7 is interposed between an inwardly facing surface 16 of the bodyside liner 5 and the absorbent core 3. Alternatively, the surge layer 7 may be located on an outwardly facing surface 18 of the bodyside liner 5. The surge layer 7 is typically constructed from highly liquid-permeable materials. Suitable materials may include porous woven materials, porous nonwoven materials, and apertured films. Some examples include, without limitation, flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. Other examples of suitable surge layers 7 are described in U.S. Pat. Nos. 5,486,166 to Ellis, et al. and 5,490,846 to Ellis, et al., which are incorporated herein in their entirety by reference thereto for all purposes.

Besides the above-mentioned components, the diaper 1 may also contain various other components as is known in the art. For example, the diaper 1 may also contain a substantially hydrophilic tissue wrapsheet (not illustrated) that helps maintain the integrity of the fibrous structure of the absorbent core 3. The tissue wrapsheet is typically placed about the absorbent core 3 over at least the two major facing surfaces thereof, and composed of an absorbent cellulosic material, such as creped wadding or a high wet-strength tissue. The tissue wrapsheet may be configured to provide a wicking layer that helps to rapidly distribute liquid over the mass of absorbent fibers of the absorbent core 3. The wrapsheet material on one side of the absorbent fibrous mass may be bonded to the wrapsheet located on the opposite side of the fibrous mass to effectively entrap the absorbent core 3.

Furthermore, the diaper 1 may also include a ventilation layer (not shown) that is positioned between the absorbent core 3 and the outer cover 17. When utilized, the ventilation layer may help insulate the outer cover 17 from the absorbent core 3, thereby reducing dampness in the outer cover 17. Examples of such ventilation layers may include breathable laminates (e.g., nonwoven web laminated to a breathable film), such as described in U.S. Pat. No. 6,663,611 to Blaney, et al., which is incorporated herein in its entirety by reference thereto for all purpose.

In some embodiments, the diaper 1 may also include a pair of ears (not shown) that extend from the side edges 32 of the diaper 1 into one of the waist regions. The ears may be integrally formed with a selected diaper component. For example, the ears may be integrally formed with the outer cover 17 or from the material employed to provide the top surface. In alternative configurations, the ears may be provided by members connected and assembled to the outer cover 17, the top surface, between the outer cover 17 and top surface, or in various other configurations.

As representatively illustrated in FIG. 2, the diaper 1 may also include a pair of containment flaps 12 that are configured to provide a barrier and to contain the lateral flow of body exudates. The containment flaps 12 may be located along the laterally opposed side edges 32 of the bodyside liner 5 adjacent the side edges of the absorbent core 3. The containment flaps 12 may extend longitudinally along the entire length of the absorbent core 3, or may only extend partially along the length of the absorbent core 3. When the containment flaps 12 are shorter in length than the absorbent core 3, they may be selectively positioned anywhere along the side edges 32 of diaper 1 in a crotch region 10. In one embodiment, the containment flaps 12 extend along the entire length of the absorbent core 3 to better contain the body exudates. Such containment flaps 12 are generally well known to those skilled in the art. For example, suitable constructions and arrangements for the containment flaps 12 are described in U.S. Pat. No. 4,704,116 to Enloe, which is incorporated herein in its entirety by reference thereto for all purposes.

The diaper 1 may include various elastic or stretchable materials, such as a pair of leg elastic members 6 affixed to the side edges 32 to further prevent leakage of body exudates and to support the absorbent core 3. In addition, a pair of waist elastic members 8 may be affixed to longitudinally opposed waist edges 15 of the diaper 1. The leg elastic members 6 and the waist elastic members 8 are generally adapted to closely fit about the legs and waist of the wearer in use to maintain a positive, contacting relationship with the wearer and to effectively reduce or eliminate the leakage of body exudates from the diaper 1. As used herein, the terms “elastic” and “stretchable” include any material that may be stretched and return to its original shape when relaxed.

The diaper 1 may also include one or more fasteners 30. For example, two flexible fasteners 30 are illustrated in FIG. 2 on opposite side edges of waist regions to create a waist opening and a pair of leg openings about the wearer. The shape of the fasteners 30 may generally vary, but may include, for instance, generally rectangular shapes, square shapes, circular shapes, triangular shapes, oval shapes, linear shapes, and so forth. The fasteners may include, for instance, a hook material. In one particular embodiment, each fastener 30 includes a separate piece of hook material affixed to the inside surface of a flexible backing.

The various regions and/or components of the diaper 1 may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In the illustrated embodiment, for example, the outer cover 17 and bodyside liner 5 are assembled to each other and to the absorbent core 3 using an adhesive. Alternatively, the absorbent core 3 may be connected to the outer cover 17 using conventional fasteners, such as buttons, hook and loop type fasteners, adhesive tape fasteners, and so forth. Similarly, other diaper components, such as the leg elastic members 6, waist elastic members 8 and fasteners 30, may also be assembled into the diaper 1 using any attachment mechanism.

Although various configurations of a diaper have been described above, it should be understood that other diaper and absorbent article configurations are also included within the scope of the present disclosure. For instance, other suitable diaper configurations are described in U.S. Pat. Nos. 4,798,603 to Meyer et al.; 5,176,668 to Bemardin; 5,176,672 to Bruemmer et al.; 5,192,606 to Proxmire et al.; and 5,509,915 to Hanson et al., as well as U.S. Patent Application Pub. No. 2003/120253 to Wentzel, et al., all of which are incorporated herein in their entirety by reference thereto for all purposes. In addition, the present disclosure is by no means limited to diapers. In fact, any other absorbent article may be formed in accordance with the present disclosure, including, but not limited to, other personal care absorbent articles, such as training pants, absorbent underpants, adult incontinence products, feminine hygiene products (e.g., sanitary napkins), swim wear, baby wipes, and so forth. Several examples of such absorbent articles are described in U.S. Pat. Nos. 5,197,959 to Buell; 5,085,654 to Buell; 5,634,916 to Lavon, et al.; 5,569,234 to Buell, et al.; 5,716,349 to Taylor, et al.; 4,950,264 to Osborn, III; 5,009,653 to Osborn, III; 5,509,914 to Osborn, III; 5,649,916 to DiPalma, et al.; 5,267,992 to Van Tillburg; 4,687,478 to Van Tillburg; 4,285,343 to McNair; 4,608,047 to Mattingly; 5,342,342 to Kitaoka; 5,190,563 to Herron, et al.; 5,702,378 to Widlund, et al.; 5,308,346 to Sneller, et al.; 6,110,158 to Kielpikowski; 6,663,611 to Blaney, et al.; and WO 99/00093 to Patterson, et al., which are incorporated herein in their entirety by reference thereto for all purposes. Still other suitable articles are described in U.S. Patent Application Publication No. 2004/0060112 A1 to Fell et al., as well as U.S. Pat. Nos. 4,886,512 to Damico et al.; 5,558,659 to Sherrod et al.; 6,888,044 to Fell et al.; and 6,511,465 to Freiburger et al., all of which are incorporated herein in their entirety by reference thereto for all purposes.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged either in whole or in part. Furthermore, those of ordinary skill in the art will appreciate that the foregoing description is by way of example only, and is not intended to limit the disclosure so further described in such appended claims.

What is claimed:

1. An absorbent article having odor control properties, the article comprising an absorbent core that includes cellulosic fibers treated with an odor control agent, the odor control agent comprising a chemical compound having a net positive charge in an amount sufficient to bind and immobilize odor producing bacteria.

2. An absorbent article as defined in claim 1, wherein the chemical compound comprises a cationic polymer, a cationic oligomer, or mixtures thereof.

3. An absorbent article as defined in claim 1, wherein the chemical compound comprises an aluminum oligomer or an aluminum salt.

4. An absorbent article as defined in claim 1, wherein the chemical compound comprises aluminum chlorohydrate, aluminum chlorohydrol, aluminum chloride, aluminum potassium sulfate, polyaluminum chloride, or mixtures thereof.

5. An absorbent article as defined in claim 1, wherein the odor control agent comprises particles coated with the chemical compound.

6. An absorbent article as defined in claim 5, wherein the chemical compound comprises an aluminum oligomer or an aluminum salt.

7. An absorbent article as defined in claim 1, wherein the odor control agent is present with the cellulosic fibers in amount from about 0.01% to about 10% by weight.

8. An absorbent article as defined in claim 1, wherein the absorbent core comprises an airformed web, the cellulosic fibers being present in the airformed web.

9. An absorbent article as defined in claim 1, wherein the article comprises an incontinent article.

10. An absorbent article as defined in claim 1, wherein the article comprises a diaper, a feminine hygiene product, a training pant, a swim pant, or an adult incontinence article.

11. An absorbent article as defined in claim 1, wherein the cellulosic fibers comprise fluff pulp fibers.

12. An absorbent article as defined in claim 1, wherein the odor control agent is bound to the cellulosic fibers.

13. An absorbent article as defined in claim 1, wherein the absorbent article includes an outer cover and a body side liner, the absorbent core being positioned in between the outer cover and the body side liner.

14. An absorbent article as defined in claim 1, wherein the absorbent core further comprises superabsorbent particles.

15. A method for forming an absorbent article with odor control properties comprising:
treating a pulp fiber sheet with an aqueous composition containing an odor control agent, the odor control agent comprising a chemical compound having a net positive charge in an amount sufficient to bind and immobilize odor producing bacteria;
fiberizing the sheet to form a plurality of individual pulp fibers;
entraining the fiber in a gaseous stream and forming an absorbent material from the gaseous stream; and
positioning the absorbent material in between a cover layer and a body side liner to form the absorbent article.

16. A method as defined in claim 15, wherein the chemical compound comprises a cationic polymer, a cationic oligomer, or mixtures thereof.

17. A method as defined in claim 15, wherein the chemical compound comprises an aluminum oligomer or an aluminum salt.

18. A method as defined in claim 15, wherein the chemical compound comprises aluminum chlorohydrate, aluminum chlorohydrol, aluminum chloride, aluminum potassium sulfate, polyaluminum chloride, or mixtures thereof.

19. A method as defined in claim 15, wherein the odor control agent is present with the pulp fibers in amount from about 0.01% to about 10% by weight.

20. A method as defined in claim 15, wherein the article comprises a diaper, a feminine hygiene product, a training pant, a swim pant, or an adult incontinence article.

21. A method as defined in claim 15, wherein the absorbent core further comprises superabsorbent particles.
